(19) United States
(12) Patent Application Publication
(10) Pub. No.: US 2024/0180663 A1
(43) Pub. Date: Jun. 6, 2024

(54) MEDICAL DEVICE DATA SYSTEM INCLUDING STERILIZATION TRAY DATA HUB AND INSTRUMENT SENSORS

(71) Applicant: Tecovan, Inc., Cedarburg, WI (US)

(72) Inventor: Michael Scherer, Cedarburg, WI (US)

(73) Assignee: Tecovan, Inc., Cedarburg, WI (US)

(21) Appl. No.: 18/522,509

(22) Filed: Nov. 29, 2023

Related U.S. Application Data
(60) Provisional application No. 63/429,575, filed on Dec. 2, 2022.

(51) Int. Cl. A61B 50/33; G16H 40/20; G16H 40/67

(52) U.S. Cl. CPC A61B 50/33; G16H 40/20; G16H 40/67

(57) ABSTRACT

A process and system for collecting and transferring sensor data, stored in a memory device of a standalone surgical instrument node sensor or a sterilization tray data hub, directly to an external or remote computer device. The process may involve storing data from one or more sensors within the surgical instrument node sensor while it is deployed within a given surgical instrument. Following the surgical procedure involving the surgical instrument node sensor, the surgical instrument containing the surgical instrument node sensor is placed within a sterilization tray featuring a sterilization tray data hub. A data link between the surgical instrument node sensor and the sterilization tray data hub is established. A secondary data link is then established between the sterilization tray data hub and a remote computer device, allowing the sensor data from both the surgical instrument node sensor and sterilization tray data hub to automatically download for remote access.

### CROSS REFERENCE TO RELATED APPLICATIONS

The present application is based on and claims priority to U.S. Provisional Patent Application Ser. No. 63/429,575 filed on Dec. 2, 2022, the disclosure of which is incorporated herein by reference.

### BACKGROUND

The present disclosure relates to a process and system for collecting and transferring sensor data, stored in a memory device of a standalone surgical instrument node sensor or a sterilization tray data hub, directly to an external or remote computer device.

### SUMMARY

This Summary is provided to introduce a selection of concepts that are further described herein below in the Detailed Description. This Summary is not intended to identify key or essential features of the claimed subject matter, nor is it intended to be used as an aid in limiting scope of the claimed subject matter.

In non-limiting examples disclosed herein, an Internet-of-Things (IoT) data hub for a medical instrument kit includes a housing and a data hub electronics module contained within the housing, wherein the IoT data hub is configured and arranged to withstand external temperatures up to 125° C. along with steam pressures of up to 208 kPa, which can occur during a sterilization procedure in an autoclave. The IoT data hub is configured and arranged to be affixed to the medical instrument kit, where the medical instrument kit is configured and arranged to receive at least one medical instrument. The data hub electronics module is designed to operate normally below a given temperature threshold and the data hub electronics module includes components that allow the electronics module to automatically enter a safe and inoperable off state while the external temperature remains above the predetermined temperature threshold. When the external temperature falls below the predetermined temperature, the components in the data hub electronics module return the data hub electronics module to a normal operating state.

The data hub of the present disclosure allows for wireless communication over short-range radio and cellular networks. A process and system are disclosed for collecting and transferring sensor data, stored in a memory device of a standalone surgical instrument node sensor or a sterilization tray data hub, directly to an external or remote computer device. The process may involve storing data from one or more sensors within the surgical instrument node sensor control unit while it is deployed within a given surgical instrument. Next, following the surgical procedure involving the surgical instrument that includes a surgical node sensor, the surgical instrument containing the surgical instrument node sensor is placed within a sterilization tray featuring a sterilization tray data hub. A data link is established between the surgical instrument node sensor and the sterilization tray data hub. A secondary data link is then established between the sterilization tray data hub and a remote computer device, allowing the sensor data from both the surgical instrument node sensor and sterilization tray data hub to automatically download to the remote computer device for remote access.

### BRIEF DESCRIPTION OF THE DRAWINGS

Examples are described with reference to the following drawing figures. The same numbers are used throughout to reference like features and components.

FIG. 1 shows a first exemplary embodiment of a sterilization tray data hub in accordance with the present disclosure having a lid and an enclosure;

FIG. 2 shows a second exemplary embodiment of a sterilization tray data hub having a hermetically sealed enclosure;

FIG. 3 shows an arrangement of components within the sterilization tray data hub;

FIG. 4 shows a perspective view of the arrangement of components within the sterilization data hub;

FIG. 5 shows one exemplary embodiment of an alternative electronic circuit board and a battery of an instrument node sensor;

FIG. 6 shows the components of the instrument node sensor of FIG. 5 within an enclosure;

FIG. 7 is a section view taken along line 7-7 of FIG. 6 showing the enclosure and instrument node sensor of FIG. 6;

FIG. 8 is a front view of one exemplary type of medical instrument having an instrument node sensor;

FIG. 9 is a sectional view taken along line 9-9 of FIG. 8, including a magnified view;

FIG. 10 is a top view of a sterilization tray having a data hub and a variety of medical instruments each having an instrument node sensor;

FIG. 11 is a top perspective view of an alternative sterilization tray having a data hub;

FIG. 12 is a flowchart depicting a process according to one exemplary embodiment of the present disclosure;

FIG. 13 is a front view of an alternative medical instrument having an instrument node sensor;

FIG. 14 is a sectional view taken along line 14-14 of FIG. 13;

FIG. 15 is a schematic diagram of the sterilization tray data hub in communication with instrument node sensors and a variety of remote devices;

FIG. 16 shows another exemplary embodiment of a sterilization tray data hub of the present disclosure having a lid and an enclosure;

FIG. 17 is a front perspective view of the sterilization tray data hub of the present disclosure having open ports in the lid;

FIG. 18 is a back perspective view of the sterilization tray data hub of the present disclosure having internal mounting threads;

FIG. 19 is a front perspective view of the sterilization tray data hub of the present disclosure with lid removed, depicting an electronic circuit board with LED indicator;

FIG. 20 is a perspective view of an electronic circuit board assembly of a sterilization tray data hub of the present disclosure with LED indicator and battery assembly;

FIG. 21 is a perspective view of an electronic circuit board and a battery of an instrument node sensor;

FIG. 22 is a perspective view of the components of the instruments sensor of FIG. 21 within an enclosure;

FIG. 23 is a section view of the enclosure of FIG. 22;

FIG. 24 is a section view of the sterilization tray data hub of FIG. 16; and

FIG. 25 is a schematic showing the electronic operating components of the data hub and the node sensor.

### DETAILED DESCRIPTION OF THE DRAWINGS

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

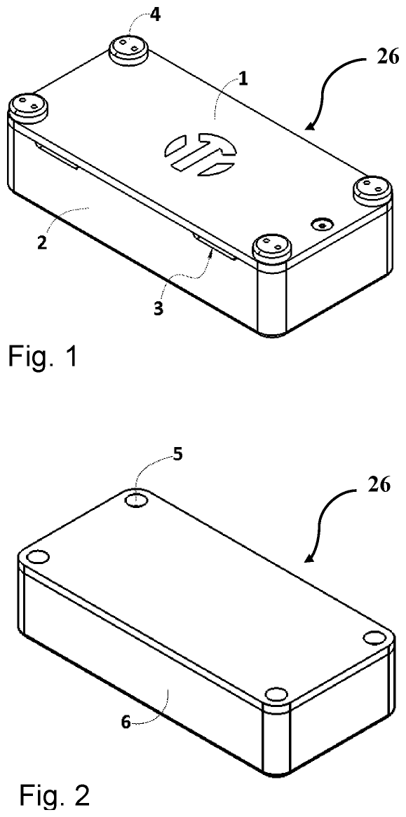

FIGS. 1 and 2 depict a first embodiment of a sterilization tray data hub 26 of the present disclosure. The data hub 26 includes a lid 1 which is removably attached to an open enclosure 2. The open enclosure 2 includes drainage slots 3 to allow steam and liquid to exit the enclosure and mounting hardware 4 for mounting additional components as will be described further herein. FIG. 2 shows an alternative sterilization tray data hub 26 that is similar to the embodiment of FIG. 1 but includes a hermetically sealed enclosure 6 having internal mounting features 5.

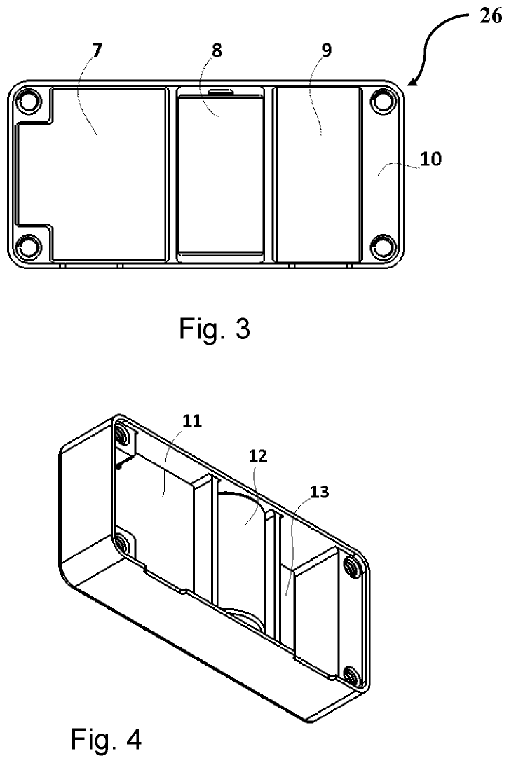

FIGS. 3 and 4 are views with the lid removed from the open enclosure and depict the arrangement of components within the sterilization tray data hub 26, including a data hub electronics module that includes an electronic circuit board 7, a battery 8, and an antenna 9, contained within the enclosure body 10. Each component is contained within a separate compartment of the enclosure body 10 such that the electronic circuit board 11, battery 12, and antenna 13, may be selectively encapsulated in a protective material such as epoxy, silicone, or conformal coating. In some embodiments, a three-compartment housing allows for encapsulation of the electronic circuit board 11, the battery 12, and the antenna 13 individually. In some embodiments, an alternative battery is formed from hermetically sealed stainless steel and thus does not need to be encapsulated in an epoxy, silicone, or conformal coating.

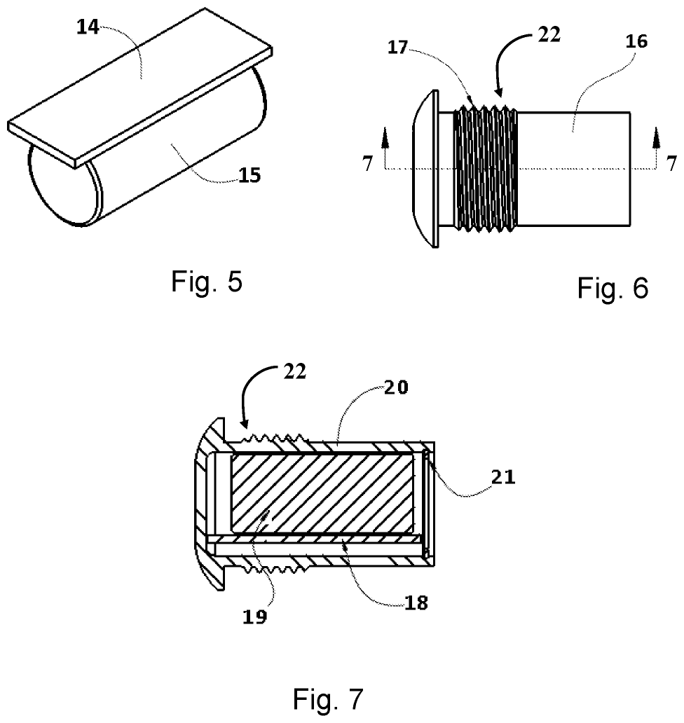

FIGS. 5 and 6 depict the components of an instrument node sensor 22. In the simplified view of FIG. 5, the instrument node sensor 22 is shown including an a node electronics module including an electronic circuit board 14 and battery 15. The circuit board 14 can include all of the required operating components of the node electronics module that are needed for the desired functions of instrument node sensor 22, which will be discussed in greater detail below. The circuit board 14 is formed from conventional circuit board material and can have the operating components mounted thereto and connected in a well-known manner. The circuit board 14 is connected to battery 15 to provide power to operate the components of the node electronics module that are included on the circuit board 14. In one contemplated embodiment, the operating components on the circuit board 14 can include a temperature actuated switch that is designed to open when the temperature exceeds a threshold value, thereby isolating the operating components of the node electronics module on the circuit board 14 from the battery power supply 15.

FIG. 6 illustrates that the battery 15 and the circuit board 14 can be contained within an enclosure 16. The enclosure 16 can include an external mounting feature 17, such as but not limited to external threads. FIG. 7 is a section view of the enclosure 16 of FIG. 6 that further illustrates another potential arrangement of components within an instrument node sensor 22, including a node electronics module including an electronic circuit board 18, battery 19, enclosure 20, and mechanical fixations 21. This alternate arrangement may be encapsulated in a protective material such as epoxy, silicone, or conformal coating. The instrument node sensor 22 is configured to be installed within a variety of medical instruments and is configured to operate normally below a given temperature threshold and automatically enters a safe and inoperable off state while the external temperature remains above the predetermined temperature threshold. As described above, one method of implementing this entry into a safe and inoperable off state is to include a temperature actuated switch that opens when the temperature exceeds the temperature threshold. When the temperature again falls below the temperature threshold, the temperature actuated switch automatically closes to provide power from the battery 19 to the components on the circuit board 18.

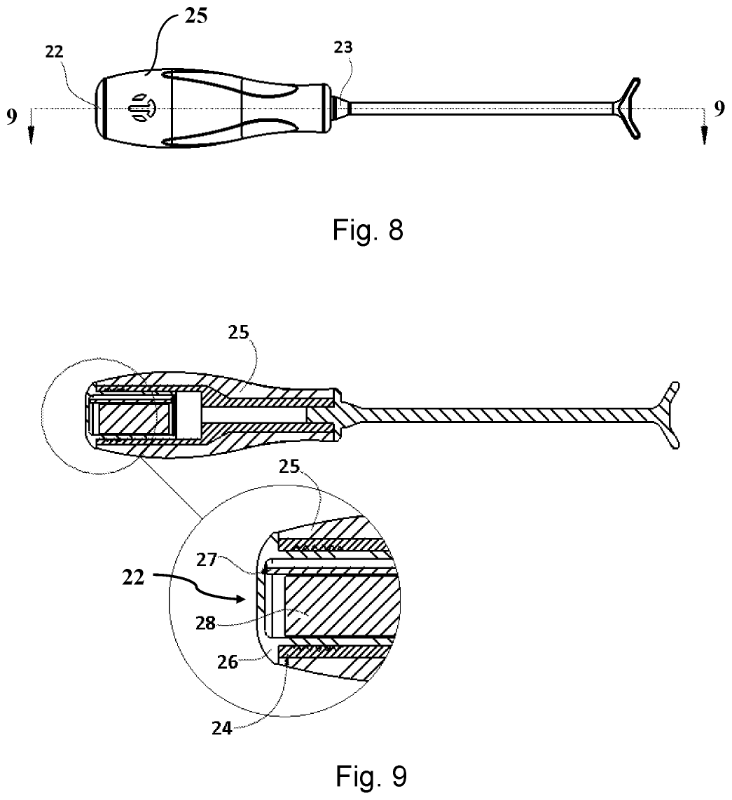

FIGS. 8 and 9 depict an example embodiment of an instrument node sensor 22 installed within an orthopedic instrument 23. In the embodiment shown, the orthopedic instrument 23 is designed to insert lumber cages during a fusion surgery. However, other types of orthopedic instruments, or other types of medical instruments, could be used while falling within the scope of the present disclosure. The orthopedic instrument 23 may be designed such that a wireless signals from the instrument node sensor 22 can be transmitted out of the orthopedic instrument by utilizing a silicone handle 25 with plastic core 24, both of which are radiolucent. This design embodiment allows for the instrument node sensor enclosure 26 to be manufactured from a radiopaque material such as stainless steel. Alternatively, the orthopedic instrument core 24 may be manufactured from a radiopaque material such as stainless steel or aluminum if the instrument node sensor enclosure 26 may be manufactured from a radiolucent material such as PPSU, PEI, or PEEK. In embodiments where the instrument node sensor 22 is installed within a surgical instrument 23 where impaction forces are of interest, such as a lumbar cage inserter, the impaction events are automatically recorded by electronic components of the node electronics module that is mounted to the circuit board of the instrument node sensor 22 when the instrument is struck during a routine surgery. This data recorded by the node electronics module of the instrument node sensor can later be analyzed to improve patient outcomes and ensure instrument lifespan compliance with regulations such as EU MDR.

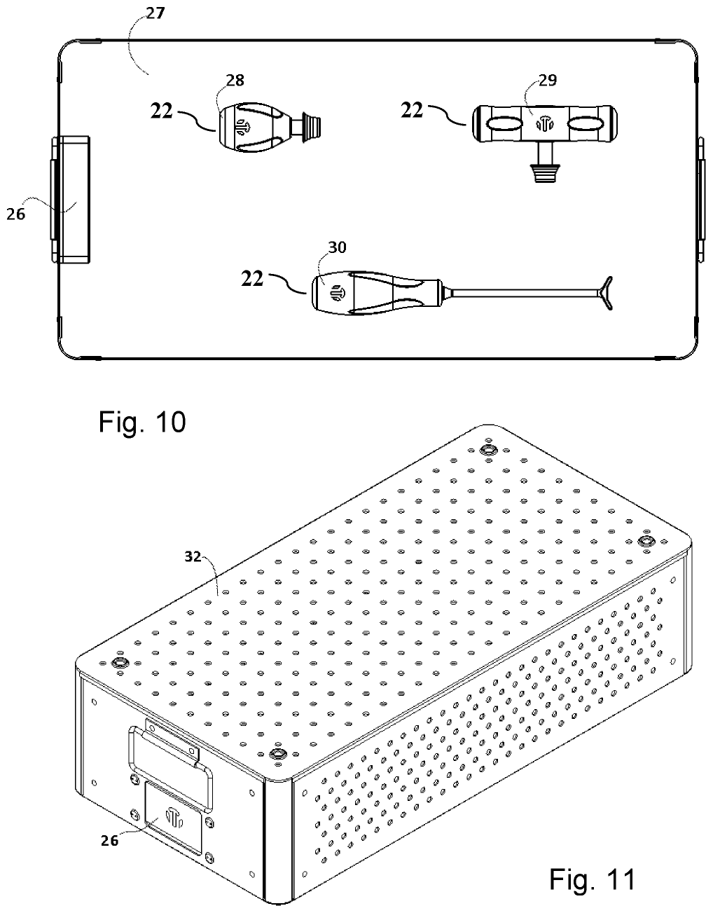

FIG. 10 depicts an example arrangement of orthopedic instruments 28, 29, and 30, each capable of containing an instrument node sensor, contained within a typical sterilization tray 27. The sterilization tray 27 also includes a sterilization tray data hub 26, shown mounted to one of the side walls of the sterilization tray 27. In the exemplary embodiment, the sterilization tray data hub 26 is mounted to the sterilization tray 32 and instrument node sensors are installed within orthopedic instruments 28, 29, 30, as shown.

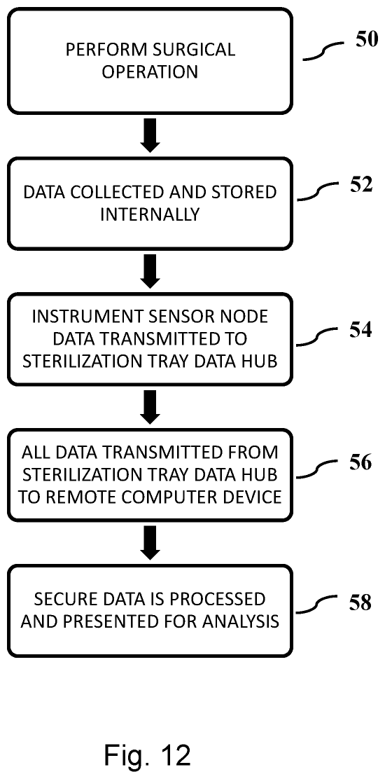

FIGS. 11 depicts an isometric view of another example sterilization tray 32 with a sterilization tray data hub 26 mounted internally. The sterilization tray data hub 26 may also be mounted externally or incorporated into the side wall of the sterilization tray 32. In both arrangements, the sterilization tray data hub 26 and instrument node sensors 22 are configured to automatically record any impaction events that the sterilization tray 32 or the orthopedic instruments 28, 29, 30 encounter. FIG. 12 illustrates one exemplary method of operation for the combination of the data hub 26 and the instrument node sensors 22, which illustrates the data collection and storage in step 52. The sterilization tray data hub 26 and the instruments sensors are configured to also automatically record data during high temperature events. In the event of the data hub 26 and instruments entering a steam autoclave oven, the data hub 26 and the instrument node sensors 22 record when a given temperature is reached and how long the sterilization tray and the instruments remains at or above the given temperature. Such a feature allows hospitals to confirm that medical devices contained on the sterilization tray were properly sterilized at the required temperature for the required period of time.

As shown in the flowchart of FIG. 12, a surgical operation is performed in step 50 utilizing the instruments that each include one of the instrument node sensors 22. The data hub 26 and any instrument node sensors 22 which may be present within the orthopedic instruments 28, 29, and 30 of the present disclosure are configured to collect and store data internally, as shown by step 52. The instrument node sensors 22 which are present within the orthopedic instruments 28, 29, 30 transmit data to the data hub 26 in step 54, which is contained within the sterilization tray 32. The data which is received by the data hub 26 is then transmitted to a remote computer device in step 56, where it is processed and presented for analysis in step 58.

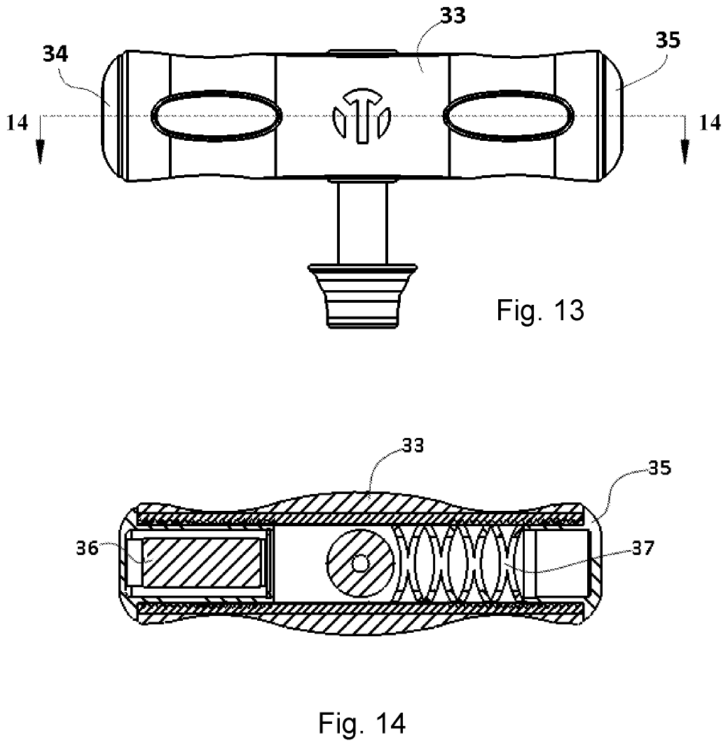

FIGS. 13 and 14 provide examples of an instrument node sensor within an orthopedic instrument, which is configured to collect and transmit data to a data hub. FIG. 13 illustrates an example embodiment of the instrument node sensor installed within a torque-limiting orthopedic instrument 33. The instrument may be outfitted with a standard cap 35 on one side and an instrument node sensor 34 on the other. FIG. 14 depicts a cross-sectional view of the torque-limiting orthopedic instrument 33 with standard mechanical torque limiting components 37 located on one side and with the instrument node sensor 36 located on the opposite side. In embodiments where the instrument node sensor 36 is installed within a surgical instrument where mechanical torque-limiting functions are of interest, the over-torque events are automatically recorded by the components of the instrument node sensor 36 when the instrument 33 is “clicked” over during a routine surgery. This data can later be analyzed to improve patient outcomes and ensure instrument lifespan compliance with regulations such as EU MDR.

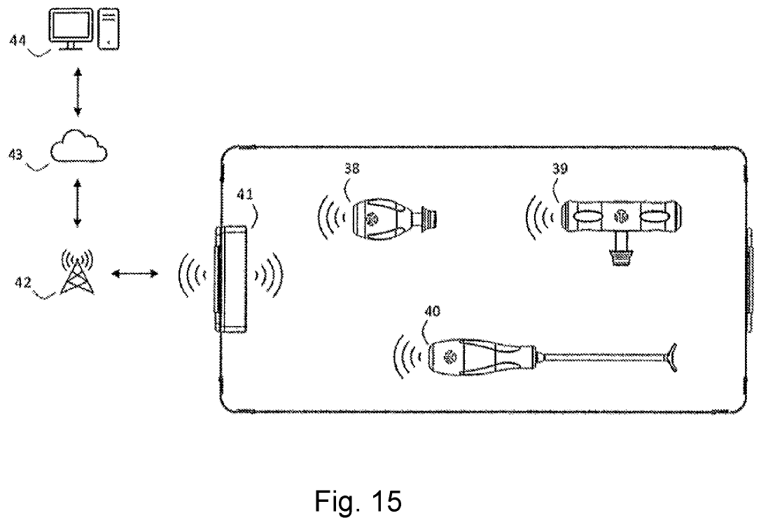

FIG. 15 is a diagram illustrating the wireless communication between several instrument node sensors 38, 39, and 40, a sterilization tray data hub 41, a cellular tower 42, a cloud server 43, and a user computer 44. The wireless communication between the sterilization data hub 41 and the remote devices 42, 43, 44 can be via short-range radio connections, such as Bluetooth, or via cellular connections. The data hub 41 can be configured to automatically turn on a Bluetooth module to search for any other compatible Bluetooth devices, such as the instrument node sensors 38, 39, 40. The instrument node sensors 38, 39, 40 can be designed to communicate with the data hub 41 utilizing any one of several different wireless communication techniques, such as but not limited to RFID, Bluetooth, Zigbee or any other communication technique that would allow for close range wireless communication. Additionally, at any given time, the data hub 41 can automatically initiate long range wireless communication, such as but not limited to cellular, WiFi, LoRaWAN or any other communication technique that would allow for long range wireless communication to transmit all gathered data to cloud servers 43 for processing and ultimately analysis by user computers 44.

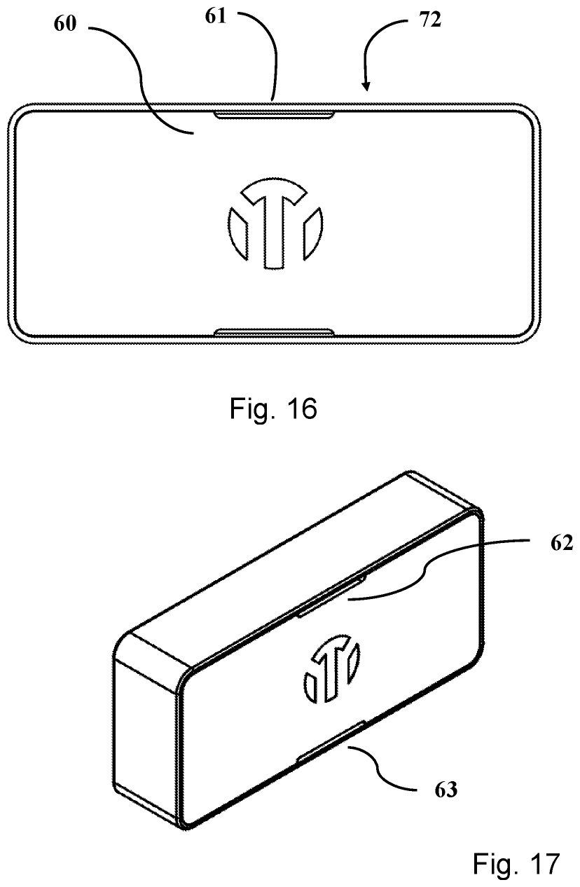

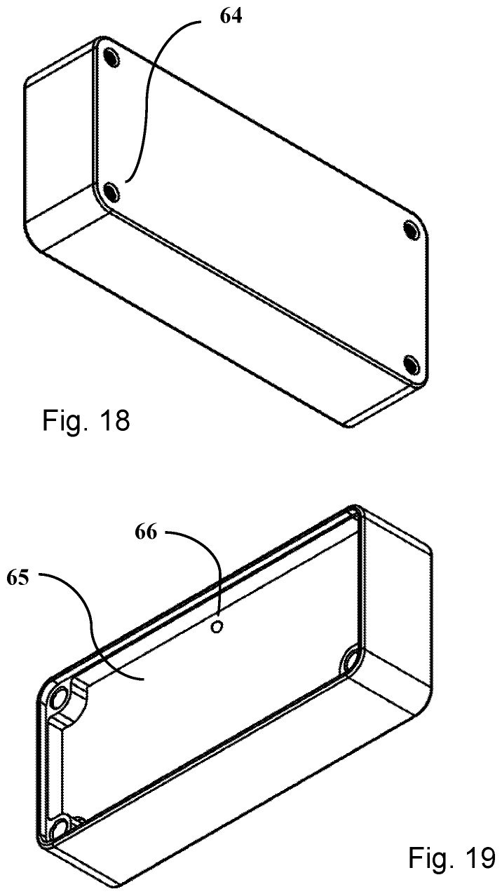

FIGS. 16-18 depict yet another alternate sterilization tray data hub 72 in accordance with the present disclosure. The data hub 72 includes a lid 60 which is removably attached to an open enclosure 61. The open enclosure 61 includes internal mounting features 64 and the lid 60 includes slots 62 and 63. The slots 62 and 63 serve dual functions of facilitating drainage of any condensed steam following an autoclave cycle as well as providing viewing ports to the indication LED 66 (FIG. 19) in embodiments where the lid 60 is made of an opaque material.

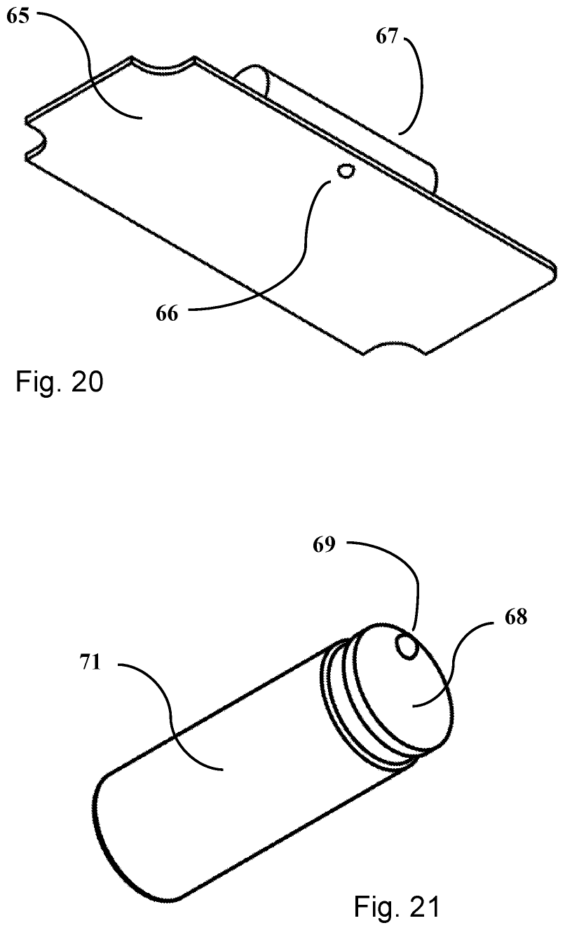

FIGS. 19 and 20 depict the arrangement of components within the alternate sterilization tray data hub 72, including an electronic circuit board 65, a LED indicator 66, and battery assembly 67. The battery assembly 67 in the embodiment illustrated can be connected to the operating components on the circuit board 65 by a temperature actuated switch (not shown) such that the operating components of the circuit board are disconnected from the battery 67 when the temperature exceeds a temperature threshold. The electronic circuit board 65 may be encapsulated in a protective material such as epoxy, silicone, or conformal coating. The LED 66 is included on the circuit board 65 and is operated to provide a visual indication as to the operational status of the data hub 72. The LED 66 can be a multi-color LED that illuminates with different colors and flashing patterns to indicate the operational status of the data hub 72.

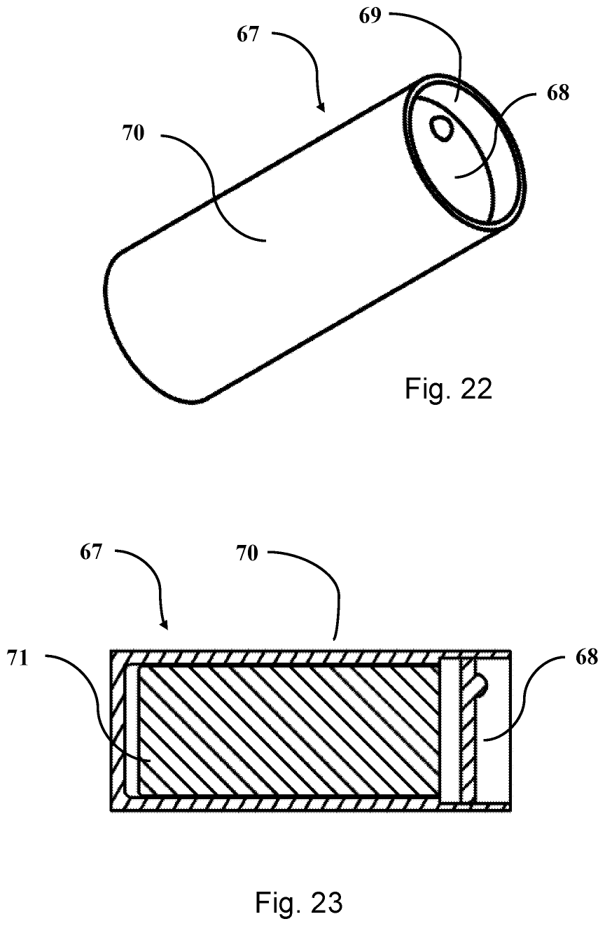

FIGS. 21 and 22 depict the components of an alternate instrument node sensor 67, including an electronic circuit board 68, an indication LED 69, and a battery 71, which can all be contained within an enclosure 70. FIG. 23 is a section view of the enclosure 70 of FIG. 22 that depicts a potential arrangement of components within the instrument node sensor 67, including an electronic circuit board 68, battery 71, enclosure 70. This arrangement may be encapsulated in a protective material such as epoxy, silicone, or conformal coating. As in the previously described embodiments, the instrument node sensor 67 is configured to be installed within a variety of medical instruments and is configured to operate normally below a given temperature threshold. The circuit board 68 can include the temperature actuated switch such that the node sensor automatically enters a safe and inoperable off state while the external temperature remains above the predetermined temperature threshold in the manner described above.

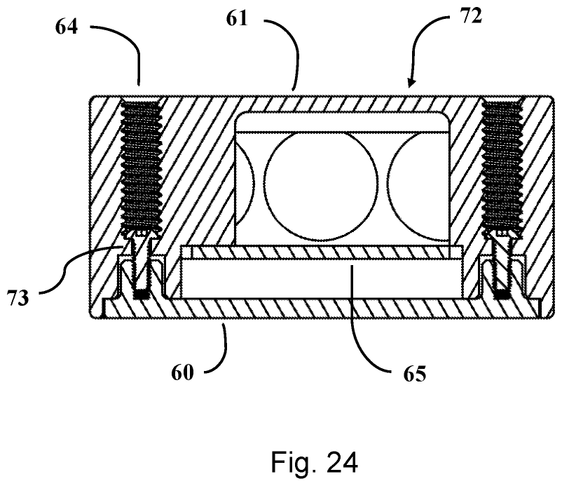

FIG. 24 is a cross sectional view of sterilization tray data hub 72 depicting a potential fixation method between housing 61 and lid 60 featuring hidden screws 73 at the base of external mounting features 64. This method may allow for electronic circuit board 65 to be encapsulated or potted in a protective material such as epoxy, silicone, or conformal coating.

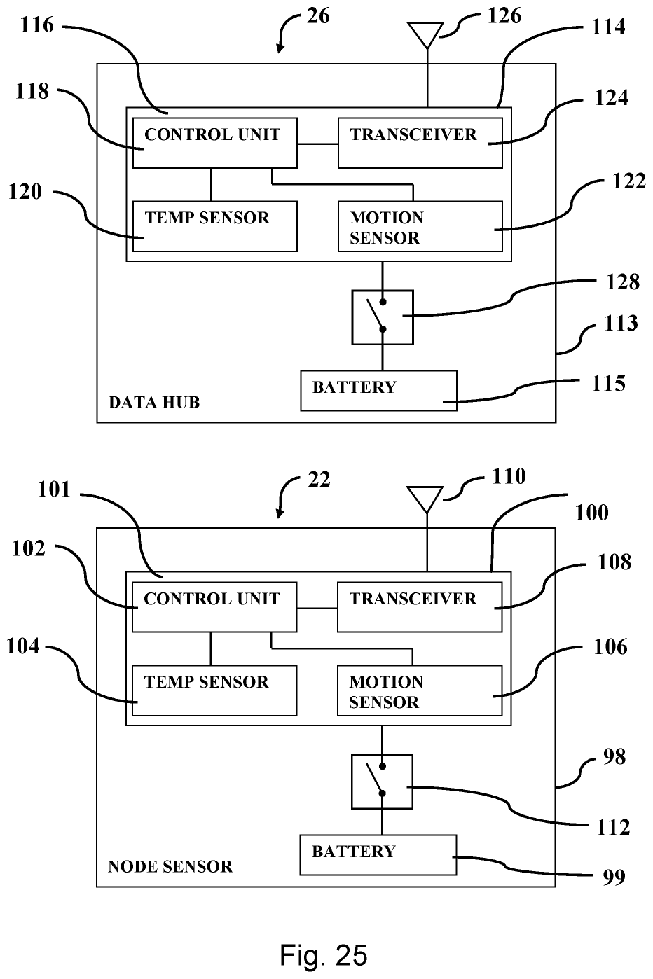

FIG. 25 is an electronic schematic diagram illustrating the operating components of both the data hub 26 and the node sensor 22. The embodiment shown is meant to illustrate one exemplary embodiment of the present disclosure and other operational embodiment could be utilized while operating within the scope of the present disclosure. In the embodiment illustrated, the node sensor 22 includes the outer enclosure 98 that is designed to receive and contain the power supply (battery 99) and node electronics module 100 that includes the circuit board 101. In the embodiment shown, the node electronics module 100 includes a control unit 102 that receives electric power from the battery 99 and is operable to control the operation and communication to and from the node sensor 22. The control unit 102 can be a microcontroller or microprocessor.

The control unit 102 is operatively connected to one or more sensors that obtain node operational data that is related to the conditions around the node sensor 22 and the operation of the medical instrument that includes the node sensor 22. In the embodiment illustrated, control unit 102 is connected to both a temperature sensor 104 and a motion sensor 106. However, other types of sensors could be used depending upon the type of medical instrument. As an example, when the node sensor 22 is used with a torque limiting instrument as shown in FIG. 13, a sensor could be used to determine over-torque events, such as an audible sensor.

The control unit 102 is further operatively connected to a transceiver 108 that is able to wirelessly transmit and receive data. The transceiver 108 is schematically shown connected to an antenna 110 that is able to support the desired type of wireless communication. The control unit 102 controls the transmission of the node operational data to the data hub 26 at desired times or upon a received request.

As was described previously, a temperature actuated switch 112 is positioned between the battery 99 and the node electronics module 100 to interrupt and disconnect the battery power supply 99 when the temperature exceeds a predetermined threshold. This disconnect protects the operating circuity of the node electronics module 100 during high temperature events, such as a steam autoclave process.

The data hub 26 is also shown schematically in FIG. 25. The data hub includes the outer housing 113 that is designed to receive and contain the power supply (battery 115) and data hub electronics module 114 that includes the circuit board 116. In the embodiment shown, the data hub electronics module 114 includes a control unit 118 that receives electric power from the battery 115 and is operable to control the operation and communication to and from the node sensor 22 and to a remote monitoring location. The control unit 118 can be a microcontroller or microprocessor.

The control unit 118 is operatively connected to one or more sensors that obtain operational data that is related to the conditions around the data hub 26. In the embodiment illustrated, the control unit 118 is connected to both a temperature sensor 120 and a motion sensor 122. However, other types of sensors could be used depending upon the desired monitoring parameters for the data hub.

The control unit 118 is further operatively connected to a transceiver 124 that is able to wirelessly transmit and receive data. The transceiver 124 is schematically shown connected to an antenna 126 that is able to support the desired type of wireless communication. The control unit 118 controls the receipt and re-transmission of the node operational data from the node sensor 22 at desired times or upon a received request.

As was described previously, a temperature actuated switch 128 is positioned between the battery 115 and the node electronics module to interrupt and disconnect the battery power supply 115 when the temperature exceeds a predetermined threshold. This disconnect protects the operating circuity of the data hub electronics module 114 during high temperature events, such as a steam autoclave process.

A typical use example for the system of the present disclosure involves the initial mounting of the sterilization tray data hub 26 to the medical sterilization tray 27. At this time, an instrument node sensor 22 is installed within a new or existing medical instrument or instruments. Once installed, both the data hub 26 and the instrument node sensors 22 automatically record any impaction events and steam autoclave cycles that the medical devices are subject to during use.

As an example, if the sterilization tray is shipped from a hospital for autoclave sterilization at a different facility, the data hub device 26 will record if it is dropped or experiences any potential damaging events during transit which could compromise the medical devices carried within the tray. In another example, if the instrument node sensor 22 is installed within a surgical instrument where impaction forces are of interest, such as a lumbar cage inserter, the impaction events are automatically recorded when the instrument is struck during a routine surgery. This data can later be analyzed to improve patient outcomes and ensure instrument lifespan compliance with regulations such as EU MDR.

In yet another contemplated embodiment, if the instrument node sensor 22 is installed within a surgical instrument where mechanical torque-limiting functions are of interest, the over-torque events are automatically recorded when the instrument is “clicked” over during a routine surgery. This data can later be analyzed to improve patient outcomes and ensure instrument lifespan compliance with regulations such as EU MDR.

Both the sterilization tray data hub 26 and the instrument node sensors 26 automatically records high temperature events. As an example, when the sterilization tray containing instruments is placed into a steam autoclave oven, the medical devices record when a given temperature is reached and how long it stayed at that elevated temperature, allowing hospitals to confirm the medical devices contained within were properly sterilized at the required temperature for the required period.

During an autoclave cycle, the data hub can turn on its Bluetooth module and search for any other compatible Bluetooth devices, such as the instrument node sensors shown in the present disclosure. This allows for ultra-low power Bluetooth data transmission from instrument node sensors to the data hub as they are known to be contained together within the sterilization oven, reducing both Bluetooth advertising time and power required for data transmission.

At a given time, the data hub can automatically initiate its cellular functionality to transmit all gathered data to cloud servers for processing and ultimately analysis by user computers.

What is claimed is:

1. A system for recording and communicating operational data from a medical instrument of a medical instrument kit to a remote monitoring location, the system comprising:
a data hub including an outer housing, a data hub electronics module and a data hub power supply, wherein the data hub electronics module and the data hub power supply are contained within the outer housing; and
a node sensor configured to be received in the medical instrument and including a node enclosure sized to receive a node electronics module and a node power supply, wherein the node electronics module is configured to communicate node operational data to the data hub,
wherein the data hub is configured to be affixed to the medical instrument kit and is operable to communicate node operational data to the remote monitoring location.

2. The system of claim 1 wherein the medical instrument kit includes a plurality of medical instruments and the system includes a plurality of node sensors each configured to be received in one of the plurality of medical instruments and operable to communicate node operational data from the node sensor to the data hub.

3. The system of claim 1 wherein the data hub and the node sensor is configured to withstand external temperature of up to 125° C. and steam pressures of up to 208 kPa.

4. The system of claim 1 wherein the node electronics module is operable to obtain node operational data that includes temperature and motion.

5. The system of claim 4 wherein the node electronics module is operable to disconnect the node power supply from the node electronics module when an external temperature is above a temperature threshold.

6. The system of claim 1 wherein the data hub electronics module is operable to obtain data hub operational data that includes temperature and motion.

7. The system of claim 6 wherein the data hub electronics module is operable to disconnect the data hub power supply from the data hub electronics module when an external temperature is above a temperature threshold.

8. The system of claim 1 wherein the data hub electronics module can communicate to the remote monitoring location by both short-range radio and cellular connections.

9. A medical instrument kit, comprising:
a one or more medical instruments;
a node sensor received in each of the medical instruments and including a node enclosure sized to receive a node electronics module and a node power supply, wherein the node electronics module is configured to obtain node operational data; and
a data hub including an outer housing, a data hub electronics module and a data hub power supply, wherein the data hub electronics module and the data hub power supply are contained within the outer housing,
wherein the node sensor is operable to wirelessly communicate the node operational data to the data hub and the data hub is operable to wirelessly communicate the node operational data to a remote monitoring location.

10. The medical instrument kit of claim 9 further comprising a sterilization tray sized to receive the one or more medical instruments during a sterilization procedure, wherein the data hub is affixed to the sterilization tray.

11. The medical instrument kit of claim 10 wherein the data hub and the node sensor are each configured to withstand external temperature of up to 125° C. and steam pressures of up to 208 kPa.

12. The medical instrument kit of claim 9 wherein the node electronics module is operable to obtain node operational data that includes temperature and motion.

13. The medical instrument kit of claim 12 wherein the node electronics module is operable to disconnect the node power supply from the node electronics module when an external temperature is above a temperature threshold.

14. The medical instrument kit of claim 9 wherein the data hub electronics module is operable to obtain data hub operational data that includes temperature and motion.

15. The medical instrument kit of claim 14 wherein the data hub electronics module is operable to disconnect the data hub power supply from the data hub electronics module when an external temperature is above a temperature threshold.

16. The medical instrument kit of claim 14 wherein the data hub communicates the node operational data to the remote monitoring location when the sensed temperature indicates a steam autoclave cycle.

17. The medical instrument kit of claim 10 wherein the node sensor communicates with the data hub when the medical instrument including the node sensor is placed in the sterilization tray.
